(19) United States
(12) Patent Application Publication
(10) Pub. No.: US 2006/0286666 A1
(43) Pub. Date: Dec. 21, 2006

(54) Methods and apparatuses for culturing stem cell using biomaterial shell

(76) Inventors: Ji Yoon Kang, Seoul (KR); Choong Kim, Seoul (KR); Jin Woo Lee, Seoul (KR); Su Kyoung Chae, Jeonju-si (KR); Dae Suk Na, Daegu-metropolitan city (KR); Byeong Kwon Ju, Seoul (KR)

Correspondence Address: ARMSTRONG, KRATZ, QUINTOS, HANSON & BROOKS, LLP;Intellectual Property Law Offices, Suite 220, 502 Washington Avenue, Towson, MD, 21204, US

(21) Appl. No.: 11/155,532

(22) Filed: Jun. 17, 2005

(51) Int. Cl. C12N 5/06; C12N 5/08; C12M 3/00

(52) U.S. Cl. 435/325; 435/289.1; 435/366

(57) ABSTRACT

Disclosed are methods and apparatuses for culturing stem cell using biomaterial shell, e.g. fertilized zebrafish chorions, which may induce formation of embryonic bodies, cell proliferation, and cell differentiation to other types effectively inside the chorions without adding any inducing agents or cell differentiation stimulants. There is provided an apparatus for culturing stem cell using biomaterial shell comprising: a microwell layer having a plurality of microwells; a microhole layer having a plurality of microholes, the microhole layer being located under the microwell layer, diameter of the microholes being smaller than diameter of the microwells and diameter of the biomaterial shell; and a flow-channel layer having a plurality of flow-channels for supplying cell medium to the microholes, the flow-channel layer being located under the microhole layer, and wherein the stem cell is encapsulated by the biomaterial shell in the microwell.

### BACKGROUND OF THE INVENTION

1. Field of the Invention

The present invention relates to methods and apparatuses for culturing stem cell. More particularly, the present invention relates to methods and apparatuses for culturing stem cell using biomaterial shell, e.g. fertilized zebrafish chorions, which may induce formation of embryonic bodies, cell proliferation, and cell differentiation to other types effectively inside the chorions without adding any inducing agents or cell differentiation stimulants.

2. Description of the Related Art

Methods of stem cell culture in the related art are classified into two dominant methods. Specifically, those methods are classified depending on how to develop embryonic bodies. Hanging drop culture method (HDC method) using high viscosity and gravity of stem cell culture medium, the first method, is to collect stem cells from bottom part of droplet type stem cell medium hanging from certain structure. The second method called liquid suspension culture (LSC) method is to collect cells chemically in conventional cell culture dishes.

The first method is popular in current biology laboratory. The method is performed in a manner that the cells collected from the bottom part interact or transfer cellular signals to each other to form an embryonic body. To protect the drop, the droplet type stem cell medium, from natural evaporation inside an incubator, PBS (phosphate-buffered saline) solution is supplied continuously into the dishes located under the drops. Formation of an embryonic body requires two through four days for cellular adhesion. Accordingly, to grow a healthy embryonic body in a drop, tiny volume of fresh culture medium should be supplied frequently and the volume of each drop should be conserved during the supply of the fresh culture medium. However, it is quite difficult to handle the drops without changing its volume seriously or falling it to the ground.

The second method of the embryonic body formation is a chemical treatment of cells in the presence of 0.5%-1% DMSO (Dimethylsulfoxide) which also works as a cell differentiation stimulant later.

After the embryonic bodies are formed by either method, they should be transferred to hydrophilic environment, called tissue culture dish, for stable embryonic body attachment. However, during this cells delivery to new dishes, they are likely to suffer from various external contaminations, for example, contamination through frequent pipetting, and the optimum concentration for cell differentiation is not likely to be maintained. Therefore, it has been known that only about 10% of embryonic bodies can be differentiated successfully to the target cells. In addition, the two methods spend extravagant culture medium for the formation of tiny embryonic bodies, of which the diameter is just about 100-200 μm.

Except the two dominant methods, a microfluidic device with a built-in multiple-well structure and several flow-channels was introduced for stable medium supply and cell handling convenience. However, such a device is nothing more than a miniaturization of current culture dishes and no additional finction of biological purposes is included. Moreover, in current cell biotechnology or cell therapy, biological materials like extracellular matrix including gelatin, collagen, and etc. consisting of various glycoproteins, are preferable to biomaterial like PMMA (poly-methylmethacrylate) or PDMS (poly-dimethylsiloxane), because cells are basically familiar to that kind of materials than artificial polymers.

### BRIEF SUMMARY OF THE INVENTION

Accordingly, the present invention has been developed to solve the above mentioned problems occurring in the related art. The object of the present invention is to provide methods and apparatuses for culturing stem cell that develop embryonic bodies and promote stem cell differentiation with high efficiency without adding any chemical additives.

In order to accomplish the objects, there is provided an apparatus for culturing stem cell using biomaterial shell comprising: microwells for arrangement of the biomaterial shell; an inlet/outlet for supplying cell medium to the stem cell; and flow-channels for connecting the inlet/outlet to the microwells, and wherein the stem cell is encapsulated by the biomaterial shell.

Preferably, the biomaterial shell may have nano pore surface structure to allow the cell medium to flow into the biomaterial shell.

Preferably, the apparatus may further comprise microholes for preventing the biomaterial shell from falling into the flow-channels, diameter of the microholes being smaller than diameter of the microwells and the biomaterial shell.

Preferably, the apparatus may further comprise a cover layer for covering the microwells and the flow-channels to protect the stem cell from medium evaporation or external contaminations.

Preferably, the biomaterial shell may be fertilized chorion.

Preferably, the stem cell may be injected by micropipette into the biomaterial shell positioned in the microwell.

Preferably, the stem cell may be P19 embryonic carcinoma (P19EC).

Preferably, the apparatus may be loaded into CO₂ incubator which contains 10% fetal bovine serum (FBS) to culture the stem cell encapsulated by the biomaterial shell.

Preferably, inner material of the biomaterial shell may be removed by injecting a jet of distilled water and ethanol through opening on the biomaterial shell.

In order to accomplish the objects, there is provided a method for culturing stem cell using biomaterial shell, comprising: injecting the stem cell into the biomaterial shell located in microwell; and loading the apparatus comprising the microwell into CO₂ incubator which contains 10% fetal bovine serum (FBS) to culture the stem cell encapsulated by the biomaterial shell.

Preferably, the method may further comprise: supplying cell medium to the biomaterial shell by inputting the cell medium through inlet/outlet connected through flow-channel to the microwell.

Preferably, the biomaterial shell may have nano pore surface structure to allow cell medium to flow into the biomaterial shell.

Preferably, said injecting the stem cell into the biomaterial shell may comprise: making an opening on the biomaterial shell; and injecting a jet of distilled water and ethanol through the opening on the biomaterial shell.

Preferably, the stem cell may be P19 embryonic carcinoma (P19EC).

Preferably, the biomaterial shell may be fertilized chorion.

Preferably, the inner material of the fertilized chorion may be removed by injecting a jet of distilled water and ethanol through opening on the fertilized chorion.

Preferably, the chorions may function as the biomaterial-shell which encapsulates stem cell. For this, fertilized zebrafish eggs may be inserted into each microwell by using a pipette. Each chorion of the zebrafish eggs may have a specific nanopore structure with a dimension of 500-700 nm in diameter with 1.5-2.0 μm intervals. Thus, cell culture medium can flow freely into the chorion through the nanopore structure, while keeping cells relatively larger than each pore, inside the chorion.

Preferably, a micropipette may pierce the fertilized zebrafish chorion and make a tiny opening on its surface. A jet of distilled water may be injected into the chorion by a micropipette. Then, the jet of distilled water may destroy and exclude york and internal embryo mass outside the chorion.

Preferably, the empty chorion may be washed with the injection of ethanol and distilled water to biologically allowable level. The biologically allowable level may be defined as a level with no bacteria left behind after washing.

Preferably, mouse P19 embryonal carcinoma stem cell in culture medium may be injected by a micropipette through the same opening after the york and internal mass were removed.

In order to accomplish the objects, there is provided an apparatus for culturing stem cell using biomaterial shell, comprising: a microwell layer having a plurality of microwells; a microhole layer having a plurality of microholes, the microhole layer being located under the microwell layer, diameter of the microholes being smaller than diameter of the microwells and diameter of the biomaterial shell; and a flow-channel layer having a plurality of flow-channels for supplying cell medium to the microholes, the flow-channel layer being located under the microhole layer, and wherein the stem cell is encapsulated by the biomaterial shell in the microwell.

Preferably, the apparatus may further comprise an inlet/outlet connected to the flow-channel layer for supplying cell medium through the flow-channels to the microholes.

Preferably, the apparatus may further comprise a cover layer for covering the microwells and the flow-channels to protect the stem cell from medium evaporation or external contaminations.

Preferably, the apparatus is loaded into CO₂ incubator which contains 10% fetal bovine serum (FBS) to culture the stem cell encapsulated by the biomaterial shell.

Preferably, the microhole layer may be manufactured by coating glass substrate with SU-8 photoresist, exposing the SU-8 photoresist layer to near UV, developing the SU-8 photoresist layer and separating the SU-8 photoresist layer from the glass substrate.

### BRIEF DESCRIPTION OF THE DRAWINGS

The above and other objects, features and advantages of the present invention will be more apparent from the following detailed description taken in conjunction with the accompanying drawings, in which:

FIGS. 1A through 1E illustrate an apparatus for culturing stem cell using biomaterial shell according to a preferred embodiment of the present invention and components thereof.

FIG. 1F illustrates fabrication procedure of microhole layer using SU-8 photoresist.

FIG. 2A illustrates that stem cell is loaded into chorion located in each microwell.

FIG. 2B illustrates a section of the apparatus illustrated in FIG. 1E.

FIG. 2C illustrates whole procedures of culturing cell inside the chorion according to a preferred embodiment of the present invention.

FIG. 3A illustrates that a tiny EB is successfully formed inside the chorion.

FIG. 3B illustrates that EB overflows the chorion through the injection opening.

### DETAILED DESCRIPTION OF THE PREFERRED EMBODIMENTS

Hereinafter, preferred embodiments of the present invention will be described with reference to the accompanying drawings. In the following description of the present invention, a detailed description of known functions and configurations incorporated herein will be omitted when it may make the subject matter of the present invention rather unclear.

Hereinafter, embryonic bodies developed by cross-adhesion between the P19 embryonic carcinoma (P19EC) stem cells will be referred to as “EBs.”

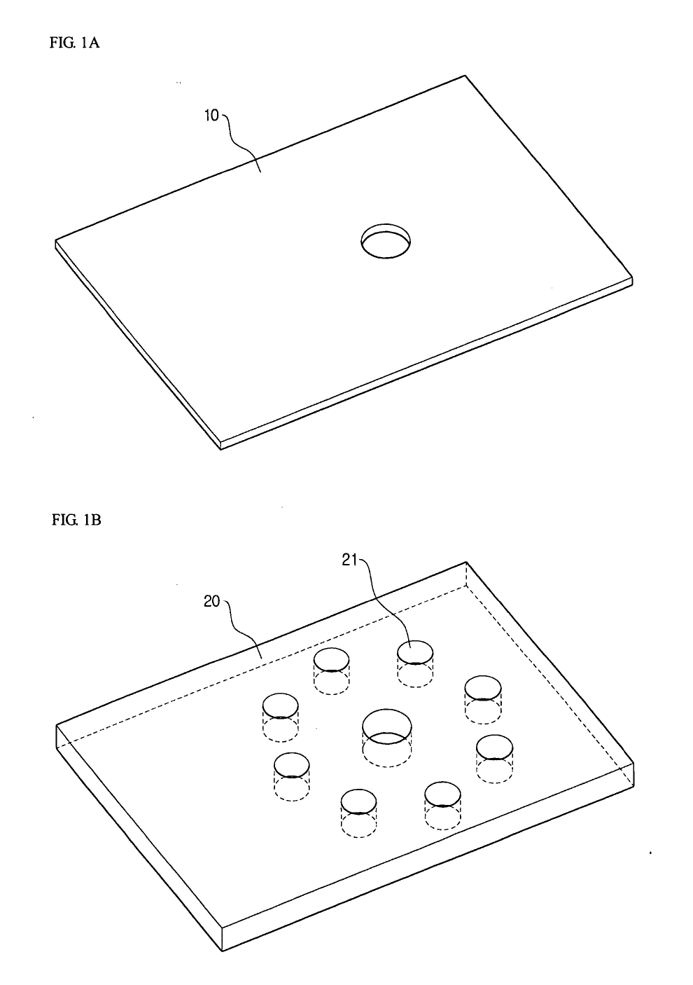

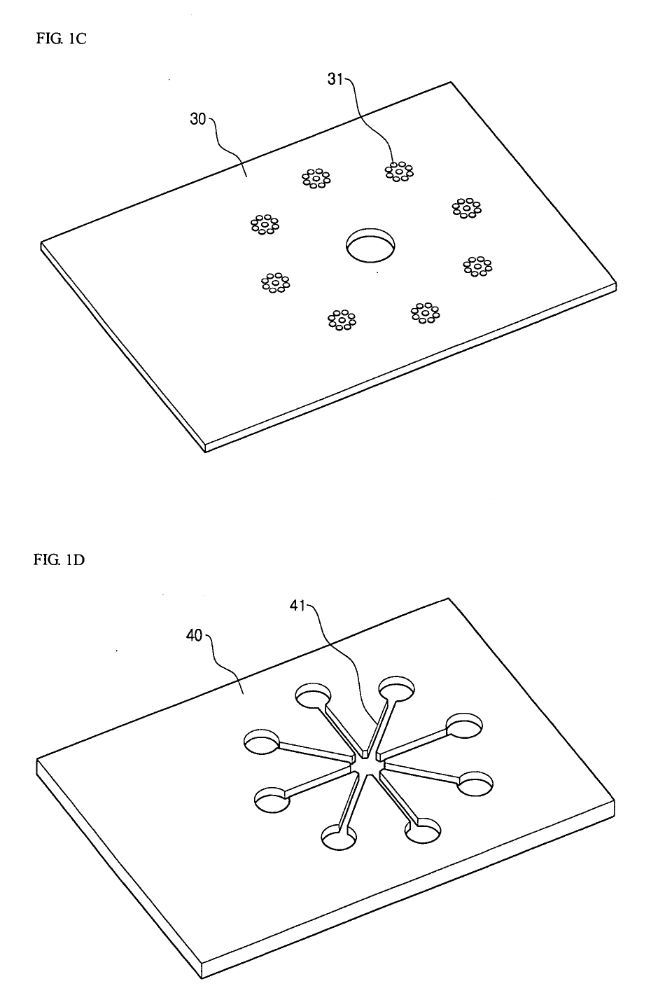

FIG. 1A through 1E illustrate an apparatus for culturing stem cell using biomaterial shell, e.g. fertilized zebrafish chorion, according to a preferred embodiment of the present invention and components thereof. The apparatus comprises: microwells (21) for arrangement of the biomaterial shell; an inlet/outlet (50) for supplying cell medium to the stem cell; and flow-channels (41) for connecting the inlet/outlet to the microwells, and wherein the stem cell is encapsulated by the biomaterial shell. On the other hand, the apparatus comprises: a microwell layer (20), a microhole layer (30), a flow-channel layer (40), and a cover layer (10). While the microhole layer(30) is made of SU-8 photoresist by using MicroElectro Mechanical Systems (MEMS), the other layers are made of polycarbonate (PC) for structural stiffiess. The role of each layer is as follows. As illustrated in FIG. 1A, the cover layer (10) functions to protect the cells from medium deficiency caused by liquid evaporation and external contaminations. The microwell layer (20) illustrated in FIG. 1B, comprises cylinder type microwells (21) for arrangement of the biomaterial shell, e.g. fertilized zebrafish chorion into which stem cell is loaded. FIG. 1D illustrates that the flow-channel layer (40) has channels (41) thereon connecting the inlet/outlet and microwells (21) to provide each microwell with culture medium. Diameter and interval of the microhole (31) are 300 μm, and 750 μm, respectively. In other words, diameter of the microhole (31) is smaller than that of the microwell (21) and that of the biomaterial shell. And, the microholes are located between the microwells and flow-channels. Thus, thanks to the microholes, it is possible for the biomaterial shell to receive supply of cell medium from the flow-channels and not to fall into the flow-channels.

FIG. 1F illustrates fabrication procedure of microhole layer using SU-8 photoresist. The microhole layer is fabricated with SU-8 photoresist. For the lithography process using a photo-mask, a soda-lime glass wafer is used as a substrate, because it shows weak adhesion to SU-8 photoresist. Thus, it is easy to detach patterned layer from the glass substrate without damaging the patterned layer.

Before coating the glass substrate with the SU-8 photoresist, the glass substrate is cleaned by the piranha etch process (H₂SO₄:H₂O₂=4:1) (S11). Then, the glass surface is dehydrated and dried by baking process at 200° C. for 5 minutes on a hotplate. Then, the glass substrate is spin-coated with SU-8 photoresist (negative type, Microchem Corporation) at 3000 rpm to form a thin film (100 μm in thickness) thereon. Then, additional baking is done at 65° C. for 10 minutes and at 95° C. for 30 minutes on a hotplate (S12). To develop a cloned pattern, the coated photoresist is exposed to near UV (350-400 nm) for 21.5 seconds after aligning a mask with a micropattern over the photoresist. Post baking is done at 65° C. for 1 minute and at 95° C. for 10 minutes (S13). Then, the photoresist is developed using SU-8 developer for 10 minutes (S14). The patterned SU-8 layer is separated from the glass substrate by sonication process in deionized water for 30 seconds and immersion in IPA (2-propannoel) for 10 seconds (S15).

The four layers of the apparatus are assembled by stacking and bonding processes using UV curing epoxy 5. The stacking sequence is as follows; from the top, the cover layer (10), the microwell layer (20), the microhole layer (30), and the flow-channel layer (40). Bonding the layers is completed by UV radiation for 1 minute.

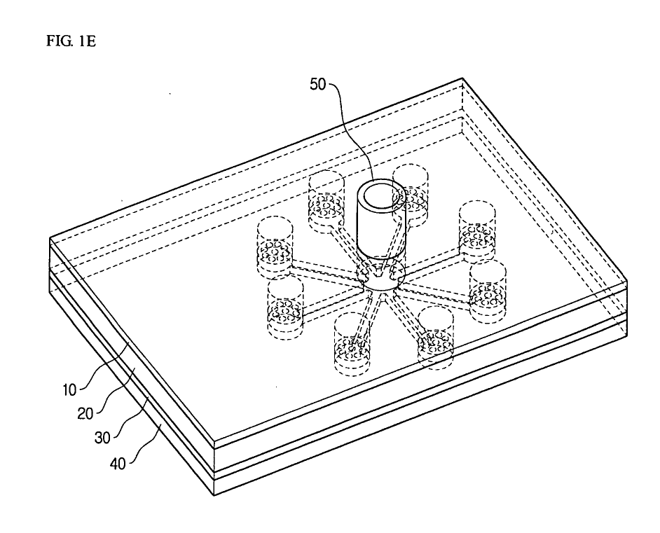

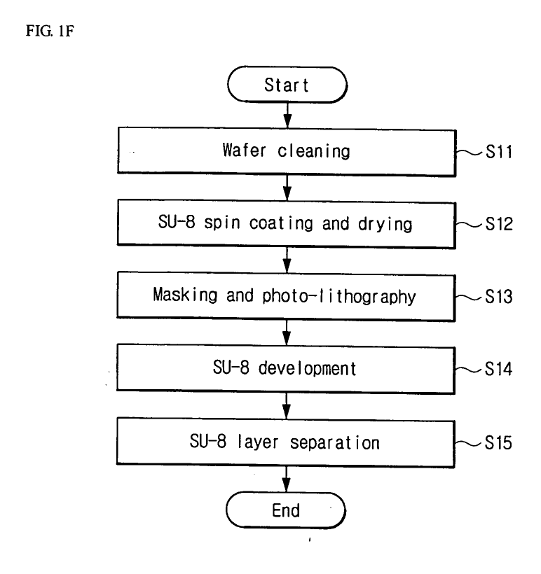

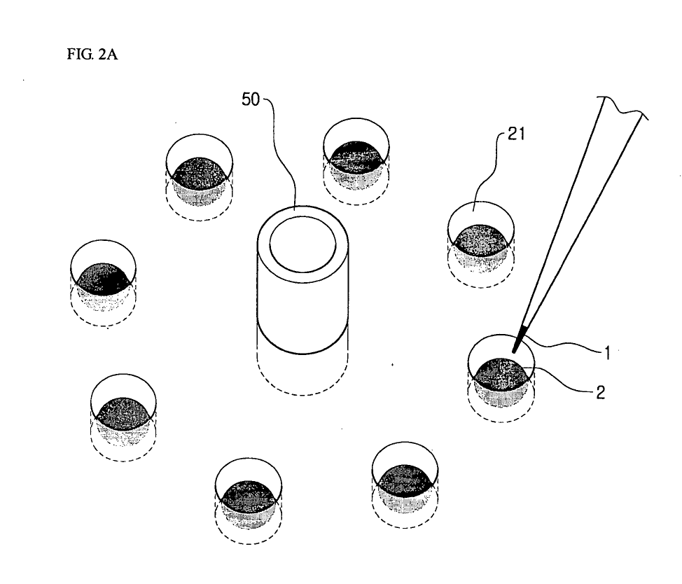

FIG. 2A illustrates that stem cell (1) is loaded into chorion (2) located in each microwell. Specifically, FIG. 2A illustrates the inlet/outlet (50), the microwell layer (20) and the microhole layer (30). Eight microwells (21) are arrayed around an inlet/outlet (50) and connected through the flow-channels (not illustrated in FIG. 2A) on the flow-channel layer (not illustrated in FIG. 2A). The culture medium is injected through the inlet/outlet (50) and supplied to microwells (21) via flow-channels. In contrast, the stem cell (1) is inserted into the microwells (21) of the microwell layer. FIG. 2B illustrates a cross-section of the apparatus illustrated in FIG 1E. It can be known from FIG. 2B that diameter of the microhole (31) is smaller than that of the microwell (21) and that of the biomaterial shell and, thus, it is possible for the biomaterial shell to receive supply of cell medium from the flow-channels (41) and not to fall into the flow-channels. The cell medium supplied to the biomaterial shell is inputted through the inlet/outlet (50).

FIG. 2C illustrates whole procedures of culturing cell inside the chorion according to a preferred embodiment of the present invention. P19EC cell may be used for this embodiment of the present invention. First, fertilized zebrafish eggs are inserted into each microwell (S31). There is a need to empty the chorion in order to use the chorion as a biomaterial shell. Thus, tiny openings are made on surface of the chorion and a jet of distilled water is injected through the openings into the chorion. For this, micropipette is used to inject the jet of distilled water into the chorion. Due to the jet of the distilled water, the yorks and inner embryo masses are destroyed and removed. Then, the chorions were sterilized by diluted ethanol (C₂H₅OH:H₂O=70:30) to repress microbial growth (S32). P19EC cell culture medium (Dulbecco's modified Eagle's medium: DMEM) is supplied through the inlet/outlet using syringe pump (S33). Then, P19EC cells (5000 cells/μl) are injected into the chorion through the openings thereon (S34). To prevent medium evaporation and external contamination, the microwell is closed by the cover layer (S35). Then, the apparatus including the zebrafish chorions are loaded into CO₂ incubator which contains 10% fetal bovine serum (FBS), and the P19EC cells in the zebrafish chorions are cultured therein for 15 days (S36).

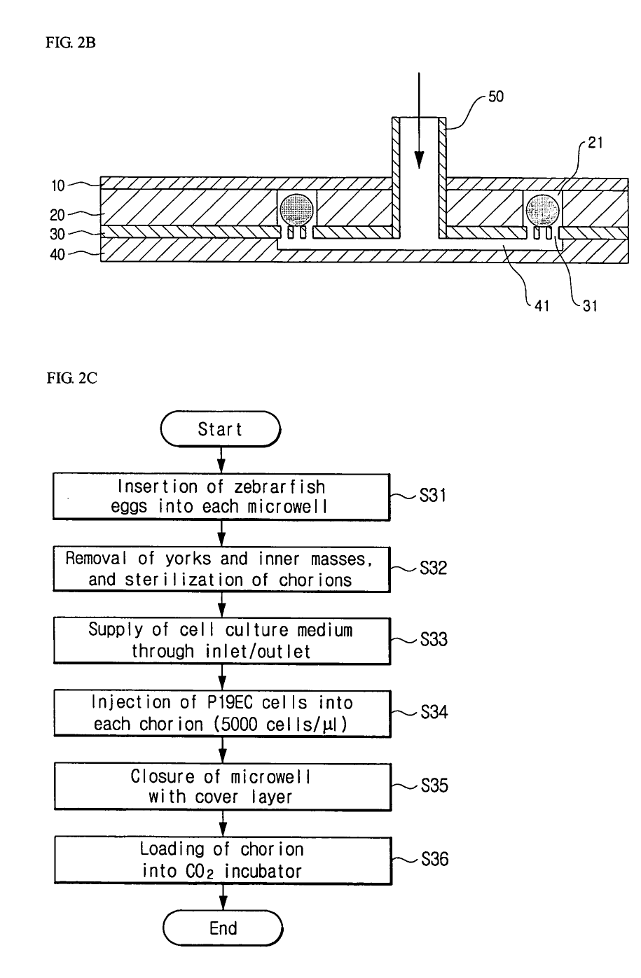

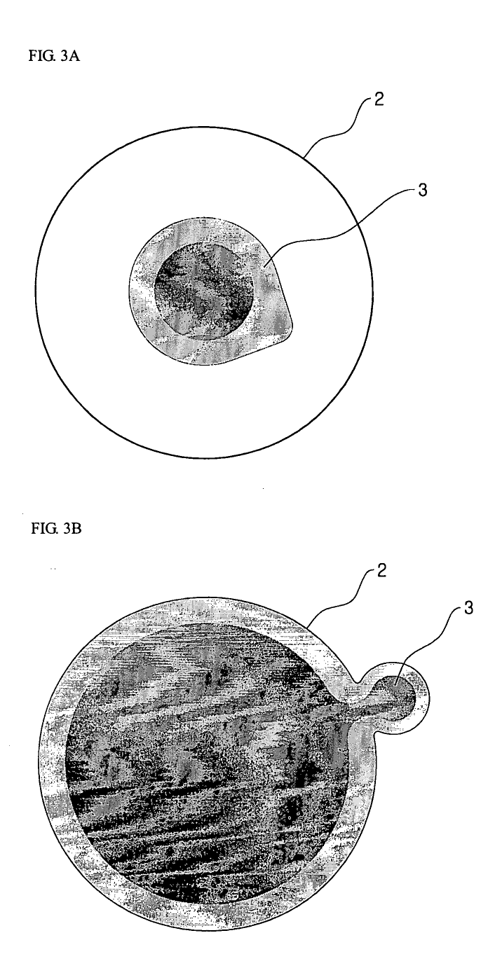

During procedures for EB growth, timely-scheduled cell lineages containing neural cells on the outer layer and beating cardiomyocytes inside EB are found; these are identified by using specific cell markers including nestin, MAP2b, GATA4 and cardiac troponin T (cTnT). As shown in FIG. 3A, a tiny EB (3) is successfully formed inside the chorion by day 4. Each EB grows continuously until it completely occupies the entire inner volume. Beyond day 10, the chorion stretches like mouse blastocyst expansion making the chorion thin. Finally, EB overflows the chorion through the injection opening at day 15 (FIG. 3B), similar to the phenomenon seen during assisted hatching. Although there is a 10% volume expansion of the chorion during cell growth, and EB overflows the chorion through a tiny surface opening of 50 μm in diameter, the chorion looks robust and no membrane splitting is observed.

For more clear observations, the stem cells are marked by using several stem cell markers, including nestin and MAP2b for neural cells, and GATA-4 and cardiac troponin T (cTnT) for cardiac cell lineages. Both nestin and MAP2b can be found extensively outside the cell mass, whereas GATA-4 and cTnT are highly marked inside the cell mass. Thus, the morphological observations and expressions demonstrate that two major cell types differentiated within the chorion in the absence of any agents for cell differentiation are neural cell and cardiomyocyte lineages, respectively.

As described above, according to the present invention, it is made possible to perform embryonic body formation, cell proliferation and cell differentiation without additional agent including differentiation stimulants.

1-9. (canceled)

10. A method for culturing stem cell using biomaterial shell, comprising:
injecting the stem cell into the biomaterial shell located in microwell; and
loading the apparatus comprising the microwell into CO₂ incubator which contains 10% fetal bovine serum (FBS) to culture the stem cell encapsulated by the biomaterial shell.

11. The method of claim 10, further comprising:
supplying cell medium to the biomaterial shell by inputting the cell medium through inlet/outlet connected through flow-channel to the microwell.

12. The method of claim 10, wherein the biomaterial shell has nano pore surface structure to allow cell medium to flow into the biomaterial shell.

13. The method of claim 10, wherein said injecting the stem cell into the biomaterial shell comprises:
making an opening on the biomaterial shell; and
injecting a jet of distilled water and ethanol through the opening on the biomaterial shell.

14. The method of claim 10, wherein the stem cell is P19 embryonic carcinoma (P19EC).

15. The method of claim 10, wherein the biomaterial shell is fertilized chorion.

16. The method of claim 15, wherein the inner material of the fertilized chorion is removed by injecting a jet of distilled water and ethanol through opening on the fertilized chorion.

17-22. (canceled)
